(19) United States
(12) Patent Application Publication
(10) Pub. No.: US 2010/0272664 A1
(43) Pub. Date: Oct. 28, 2010

(54) Alpha or Beta Hydroxy Acid Adducts of Oil

(75) Inventors: Jairajh Mattai, Piscataway, NJ (US); Ravi Subramanyam, Belle Mead, NJ (US)

Correspondence Address: COLGATE-PALMOLIVE COMPANY, 909 RIVER ROAD, PISCATAWAY, NJ, 08855, US

(73) Assignee: COLGATE-PALMOLIVE COMPANY, New York, NY (US)

(21) Appl. No.: 12/373,368

(22) PCT Filed: Nov. 20, 2008
(86) PCT No.: PCT/US2008/084173

§ 371 Date: Jan. 12, 2009

Related U.S. Application Data
(60) Provisional application No. 60/990,818, filed on Nov. 28, 2007.

(51) Int. Cl. A61K 8/362; C07C 57/02; C11D 17/00; A61Q 19/10

(52) U.S. Cl. 424/65; 554/122; 510/152; 510/158

(57) ABSTRACT

A composition of matter comprising an oil adduct of an oil capable of undergoing an adduct reaction and at least one addition material chosen from an alpha hydroxy acid and a beta hydroxy acid.

### CROSS-REFERENCE TO RELATED APPLICATIONS

This application claim priority to U.S. Provisional Patent Application No. 60/990,818, filed on 28 Nov. 2007, which is incorporated herein by reference.

### BACKGROUND OF THE INVENTION

Providing moisture to skin, hair, or nails has been the goal of many products to prevent dryness and/or promote moisturization. Some products have used materials as a barrier to prevent moisture from escaping. Other products use materials to attract moisture to the skin.

It would be desirable to provide a composition that could provide a desired level of moisture along with desired effects on skin, hair, and/or nails.

### BRIEF SUMMARY OF THE INVENTION

A composition of matter comprising an oil adduct of an oil capable of undergoing an adduct reaction and at least one addition material chosen from an alpha hydroxy acid and a beta hydroxy acid.

### DETAILED DESCRIPTION OF THE INVENTION

As used throughout, ranges are used as shorthand for describing each and every value that is within the range. Any value within the range can be selected as the terminus of the range.

The present invention is directed to a composition including an oil adduct. The oil adduct may be synthesized from an oil and an alpha or beta hydroxy acid. In some embodiments, the oil adduct may be added to personal care products, and may exhibit moisturization benefits.

### Oil

In sonic embodiments, the oil adduct is synthesized from an oil which is capable of undergoing an adduct reaction. In some embodiments, the oil contains a hydroxyl group. In some embodiments, the oil is castor oil. Castor oil is one of three triglycerides that contain principally one fatty acid; it is about 90% ricinoleic acid, or 12-hydroxyoleic acid. The other two are tung oil, which is about 80% eleostearic acid, and oiticica oil, which is about 80% licanic acid. The hydroxyl group of the ricinoleic acid reacts like a typical secondary alcohol, i.e. it can be eliminated or esterified.

### Addition Material

The addition material can be any alpha or beta hydroxy acid. In one embodiment, the acid is at least one acid chosen from lactic acid, glycolic acid, lactobionic acid, carnitine, and salicylic acid.

By using an alpha hydroxy acid or a beta hydroxy acid, an additional benefit beyond moisturization can be obtained. When the composition contacts the skin, the alpha hydroxy acid or a beta hydroxy acid can be liberated from the molecule by skin enzymes, such as esterases. The free acid then can act as an exfoliating agent in combination with the moisturization effect.

### Method of Making

According to some embodiments, the ethoxylated oil is reacted with the alpha or beta hydroxy acid. An adduct reaction that can yield a high purity adduct can be found in U.S. Pat. No. 6,225,485 to Bertz et al.

### Use

In some embodiments, the oil adduct exhibits moisturization benefits. In some embodiments, the oil adduct is included in a personal care product. A personal care product containing the oil adduct may exhibit moisturization benefits. An oil adduct of the present invention may be added to any personal care product, including but not limited to body washes, bar soaps, liquid soaps, lotions, shampoo, conditioners, antiperspirants/deodorants, and cosmetics.

### Cleansing Composition

In some embodiments, an oil adduct of the present invention is added to a cleansing composition, such as a body wash or shower gel. In some embodiments, a cleansing composition may include about 0.1 to about 5 weight % oil adduct, about 0.1 to about 3 weight % oil adduct, or about 0.1 to about 1 weight % oil adduct.

### Optional Moisturizing Agents

In addition to an oil adduct, cleansing composition may include a moisturizing agent chosen from a hydrolyzed keratin, hydroxyethyl urea, and/or a quaternized nitrogen moisturizing agent.

In one embodiment, a hydrolyzed keratin is present in the composition. Any suitable hydrolyzed keratin can be included in the composition. In one embodiment, the hydrolyzed keratin comprises an extract of goat hair. In one embodiment, the goat hair is cashmere. The hydrolyzed keratin can be present in the composition in any desired amount to give a desired level of moisturization. In one embodiment, the hydrolyzed keratin is present in an amount of greater than 0 to about 0.005% by weight. In another embodiment, the hydrolyzed keratin is present in an amount of about 0.0005 to about 0.005% by weight. In another embodiment, the hydrolyzed keratin is present at about 0.0015% by weight.

In one embodiment, hydroxyethyl urea is present in the composition. The hydroxyethyl urea can be present in the composition in any desired amount to give a desired level of moisturization. In one embodiment, the hydroxyethyl urea is present in an amount of greater than 0 to about 13% by weight. In one embodiment, the hydroxyethyl urea is present at about 6% by weight.

In one embodiment, a quaternary nitrogen moisturizing agent is present in the composition. The quaternary nitrogen moisturizing agent is a moisturizing agent that contains a quaternary nitrogen in its structure. Examples of quaternary nitrogen moisturizing agents include, but are not limited to, hydroxypropyl bis-hydroxyethyldimonium chloride (available as COLAMoist™ 200 from Colonial Chemicals, Inc.), which has a structure that is described in U.S. Pat. No. 6,869,977, a choline salt (which is described in U.S. Pat. Nos. 6,475,965 and 6,265,364), carnitine, and combinations thereof. Naturally occurring carnitine is L-carnitine. The quaternary nitrogen moisturizing agent can be present in the composition in any desired amount to give a desired level of moisturization. In one embodiment, the quaternary nitrogen moisturizing agent is present in an amount of greater than 0 to about 5. In another embodiment, the quaternary nitrogen moisturizing agent is present in an amount of about 0.1 to about 1% by weight. In another embodiment, the quaternary nitrogen moisturizing agent is present at about 1% by weight.

Additionally, glycerin may be included in the composition in combination with the moisturizing agent. The glycerin can be included in any desired amount to provide a desired level of moisturization. In one embodiment, the glycerin is present in an amount of greater than 0 to about 8% by weight. In other embodiments, the glycerin can be present at about 6% by weight or about 1.5% by weight.

The composition may also contain creatine. Creatine can be used to support the energy cycle in skin cells. Creatine can be included at any desired amount to achieve any desired level of energy support in cells. In one embodiment, the creatine is present in the composition in an amount of greater than 0 to about 2% by weight.

### Surfactants

The cleansing compositions also include one or more anionic surfactants, amphoteric surfactants, nonionic surfactants, cationic surfactants, and combinations thereof. Those of ordinary skill in the art will be aware of suitable surfactants and other additives readily identifiable from the International Cosmetic Ingredient Dictionary and Handbook, 10th ed., (2004). Surfactants can be included in any desired amount. In one embodiment, surfactants are present in the composition in an amount of greater than 0 to about 40% by weight. In one embodiment, the surfactants are present in an amount of about 1 to about 40% by weight. In one embodiment, surfactants are present in the composition in an amount of about 5 to about 40% by weight. In one embodiment, the surfactants are present in an amount of about 1 to about 10% by weight.

A variety of anionic surfactants can be utilized in the moisturizing body wash composition including, for example, long chain alkyl (C₆-C₂₂) materials such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, for example, ethoxylated, although alkoxylation is not required. These surfactants are typically highly water soluble as their sodium, potassium, alkyl and ammonium or alkanol ammonium containing salt form and can provide high foaming cleansing power. Other equivalent anionic surfactants may be used. In one embodiment, the anionic surfactant comprises sodium laureth sulfate, sodium pareth sulfate, and combinations thereof. Anionic surfactants can be included in any desired amount. In one embodiment, anionic surfactants are present in the composition in an amount of greater than 0 to about 15% by weight. In one embodiment, anionic surfactants are present in an amount of about 6 to about 8% by weight.

Amphoteric surfactants may also be included in the composition. These surfactants are typically characterized by a combination of high surfactant activity, lather forming and mildness. Amphoteric surfactants include, but are not limited to derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate. Examples of such compounds include sodium 3-dodecyaminopropionate, sodium 3-dodecylaminopropane sulfonate, N-alkyl taurines and N-higher alkyl aspartic acids. Other equivalent amphoteric surfactants may be used. Examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine, sulfobetaines such as coco dimethyl sulfopropyl betaine, stearyl dimethyl sulfopropyl betaine, amido betaines, amidosulfobetaines and the like. Betaines having a long chain alkyl group, such as coco, may be particularly useful as are those that include an amido groups such as the cocamidopropyl and cocoamidoethyl betaines. Amphoteric surfactants can be included in any desired amount. In one embodiment, amphoteric surfactants are present in the composition in an amount of greater than 0 to about 15% by weight. In one embodiment, the amphoteric surfactants are present in the composition in an amount of about 4 to about 6% by weight.

Examples of nonionic surfactants include, but are not limited to, polysorbate 20, long chain alkyl glucosides having C₈-C₂₂ alkyl groups; coconut fatty acid monoethanolamides such as cocamide MEA; coconut fatty acid diethanolamides, fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (for example the PLURONIC™ block copolymers commercially available from BASF); fatty acid alkylolamides, (fatty acid amide polyethylene glycols); N-alkyl-, N-alkoxypolyhydroxy fatty acid amides; sucrose esters; sorbitol esters; polyglycol ethers; and combinations thereof. Nonionic surfactants can be included in any desired amount. In one embodiment, nonionic surfactants are present in the composition in an amount of greater than 0 to about 3% by weight. In one embodiment, nonionic surfactants are present in the composition in an amount of about 0.5 to about 1.5% by weight.

Cationic surfactants can also be included in the composition. Examples of cationic surfactants include, but are not limited to any quaternium or polyquaternium compound. Cationic surfactants can be included at any desired level. In one embodiment, cationic surfactants are present in the composition in an amount of greater than 0 to about 2% by weight. In one embodiment, cationic surfactants are present in the composition in an amount of about 0.1 to about 0.3% by weight.

Many additional surfactants are described in McCUTCHEON'S DETERGENTS AND EMULSIFIERS (1989) and other reference materials that are well known to those of ordinary skill in the art.

### Oils

Skin compatible oils can be included in the composition. Skin compatible oils include a range of liquid hydrocarbons, for example, linear and branched oils such as liquid paraffin, squalene, squalane, mineral oil, low viscosity synthetic hydrocarbons such as polyalphaolefins, commercially available from ExxonMobil under the trade name PURESYN PAO and polybutene under the trade name PANALANE™ or INDOPOL™. Light (low viscosity) highly branched hydrocarbon oils may also be suitable in some instances. Other useful skin compatible oils may be silicone based, for example, linear and cyclic polydimethyl siloxane, organo functional silicones (alkyl and alkyl aryl), and amino silicones.

### Additional Materials

In other embodiments, the composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5% by weight); foaming agents, for example decyl glucoside, and/or their equivalents (0 to 3% by weight); glyceryl esters and derivatives, for example glycol distearate, and/or their equivalents (0 to 3% by weight); sequestrants, for example, tetrasodium EDTA, and/or their equivalents (0 to 2% by weight); biocides, for example, Triclosan (2,4,4′-trichloro-2′-hydroxydiphenyl ether), DMDM hydantoin, formaldehyde and/or imidazolidinyl urea, and/or their equivalents (0 to 2% by weight); organic acids, for example, citric acid and/or formic acid and/or their equivalents (0 to 2% by weight); viscosity modifiers (0 to 2% by weight); fragrances and/or perfumes (0 to 5% by weight); preservatives, for example, phenoxyethanol, formaldehyde solution, parabens, pentanediol or sorbic acid (0 to 2% by weight); pearlizing agents, for example, glycol distearic esters, such as ethylene glycol distearate, but also fatty acid monoglycol esters (0 to 3% by weight); stabilizers, for example, metal salts of fatty acids, such as e.g. magnesium stearate, aluminum stearate and/or zinc stearate (0 to 2% by a weight); and dyes and pigments that are approved and suitable for cosmetic purposes.

### Water

Water may be included in the composition. Water can be included in an amount of greater than 0 to about 95% by weight. In one embodiment, water is present at about 50% to about 90% by weight.

### Thickening Agent

In one embodiment, a cleansing composition also utilizes, as a thickening agent, a blend of PEG-150 distearate and PPG-2 hydroxyethyl cocamide for countering a decrease in viscosity associated with the concentrations of moisturizing agents utilized in some embodiments of the moisturizing body wash composition. This blended thickening agent allows the composition to achieve viscosities beyond those that could be achieved with conventional thickening agents, for example sodium chloride alone, and is able to achieve suitable viscosities at relatively low concentrations. The relatively low concentrations used to achieve the desired viscosities are also advantageous with respect to manufacturing processes that may be employed to manufacture the moisturizing body wash composition, thereby reducing the need for larger equipment or modifications and the capital expenditure associated with manufacturing the moisturizing body wash composition if other thickening agents were used. The PEG-150 distearate and the PPG-2 hydroxyethyl cocamide can be present in any amount to achieve a desired viscosity. In one embodiment, the amount of PEG-150 distearate in the composition is 0 to about 2% by weight. In one embodiment, the amount of PPG-2 hydroxyethyl cocamide in the composition is 0 to about 2% by weight. In one embodiment, the weight ratio of the PEG-150 distearate to the PPG-2 hydroxyethyl cocamide can be about 3:1 to about 1:3. In one embodiment the PEG-150 distearate and the PPG-2 hydroxyethyl cocamide are each present at 0.0225% by weight. The PEG-150 distearate and the PPG-2 hydroxyethyl cocamide are available as a mixture from Uniqema under the trade name PROMIDIUM™ LTS.

The cleansing composition can be used to moisturize skin, hair, and/or nails. The composition may be applied to skin, hair, and/or nails. If the composition is a rinse off composition, the composition is rinsed off. The composition can be left on for any desired amount of time. The composition can be included in any product that contacts skin, including the oral cavity hair, and/or nails. The composition can be used on humans or other animals. The composition can be in the form of a body wash, a shower gel, a hand wash, a soap bar, a shampoo, a conditioner, a dishwashing liquid, a skin lotion, a sunscreen, a bubble bath, an oral care product, a dentifrice, a toothpaste, a mouthwash, an antiperspirant, a deodorant, or a foot soak.

The composition can also be used to apply a substance to a substrate. The substance is included in the composition, and the composition is applied to a substrate. The substrate can be any desired substrate. In one embodiment, the substrate can be skin, hair, and/or nails. The substance can be any substance that is attracted to the composition. In one embodiment, the substance is chosen from fragrances, sunscreen, pigments, insect repellents, and/or hydrophobic materials.

### Bar Soap

In some embodiments, an oil adduct of the present invention is added to bar soap formulations. In some embodiments, a bar soap contains about 0.1 to about 5% by weight of the oil adduct.

A soap bar of the present invention may include any conventional soap bar materials. Examples of bar soap formulations and methods of making are disclosed by EP 0463912B1 and EP 1356018B1. The compositions generally contain about 45 weight % to about 95 weight %, or in some embodiments about 55 weight % to about 88 weight %, of soap, i.e. soluble alkali metal salt of a C₈ to C₂₄, or in some embodiments C₁₀ to C₂₀ fatty acid. In some embodiments, free fatty acids of fats or oils of the same general carbon content as the fatty acid component of the soap may be incorporated in the soap composition. A bar soap may contain about 0.5 weight % to about 20 weight %, or in some embodiments about 1 weight % to about 10 weight %, or in still other embodiments about 2 weight % to about 8 weight % free fatty acid.

In some embodiments, the bar soap compositions contain about 0 weight % to about 45 weight %, or in some embodiments about 0 weight % to about 35 weight % of a non-soap synthetic anionic surfactant.

### Anionic Surfactants

Examples of suitable non-soap anionic surfactants include, but are not limited to the salts of higher fatty alcohol sulfates wherein the higher fatty alcohol is generally of about 10 to 18 carbon atoms and which may be ethoxylated with about 0 to about 10 moles ethylene oxide, or in some embodiments about 0 to about 5 moles ethylene oxide, such as 2 or 3 moles ethylene oxide per mole of fatty alcohol. Other useful anionic surfactants include the sulfated and sulfonated detergents, such as the higher fatty acid monoglyceride sulfates of 10 to 18 carbon atoms in the fatty acid moieties, the paraffin sulfonates, olefin sulfonates, and branched and linear alkyl benzene sulfonates of 10 to 18 carbon atoms in the lipophilic groups thereof. In some embodiments, it is preferred to incorporate anionic surfactants which are most biodegradable. In some embodiments, these anionic surfactants are employed as their water-soluble salts, such as sodium salts. The cation portion may also be one or more of potassium, ammonium, magnesium, and calcium or an organic cation, such as mono-, di-, or triethanolamine. In some embodiments, sodium salts constitute more than 50%, or in some embodiments more than 75%, or in some embodiments about 100% of the cation of the anionic detergent surfactants.

In some embodiments, a bar soap contains glycoside surfactants. In some embodiments, the benefits of the glycoside surfactants become noticeable when used in an amount of about 1.5 wt % to about 2.3 wt %, or about 1.8 to 2.0 wt % based on the total composition. However, even greater benefits are often observed when the glycoside surfactant is used in amounts as high as about 20% by weight of the composition, especially up to about 12 or about 15%, such as 6%, 8%, 10%, etc.

### Water

The amount of moisture present in the soap bar compositions is not critical and may be selected depending upon the final desired properties of the product as is well known to those skilled in the art. Generally, amounts of water of about 10% to about 26%, or about 15% to 24%, by weight of the composition, will be present. In the range of moisture of about 17% to 22%, the products tend to be more highly translucent to nearly transparent. However, this range may vary depending on the content of free fats, fatty acids or oils in the composition which tend to make the soap bar product less translucent, i.e. let less light pass through the bar.

### Additional Materials

In some embodiments, a bar soap contains skin conditioning components, processing aids, anti-bacterial agents and sanitizers, dyes, perfumes, pearlescent agents, coloring agents, combinations thereof, and the like.

Materials to facilitate the preparation of bars can also be present. Thus, glycerin, for example, can be added to the crutcher or amalgamator in order to facilitate processing. Glycerin, if present, generally comprises about 0.2% to about 10% by weight of the finished bar. Additionally, emulsifiers such as polyglycerol esters (e.g. polyglycerol monostearate), propylene glycol esters and other chemically stable nonionic materials may be added to the bars to help solubilize various components, such as skin conditioning agents, such as sorbitan esters. Alkali metal citrates are also valuable herein as plasticizers.

Conventional anti-bacterial agents and sanitizers may be present. Typical anti-bacterial sanitizers include, for example, 3,4-di- and 3′,4′,5-tribromosalicyl-anilides, 4,4′-dichloro-3-(trifluoromethyl)carbanalide; 3,4,4′-trichloro-carbanalide and mixtures of these materials. If present, anti-bacterial agents and sanitizers generally comprise about 0.5% to about 4% by weight of the finished bar.

Various emollients and skin conditioning agents may also be present, for example, sorbitan esters, such as those described in U.S. Pat. No. 3,988,255, lanolin, cold cream, mineral oil, isopropyl myristate, and similar materials. When present, such emollients and skin conditioning agents generally comprise about 0.5% to about 5% by weight of the bar.

The bar soaps may also contain an electrolyte. Suitable electrolytes include, for example, sodium chloride, potassium chloride, potassium carbonate, dipotassium monohydrogen orthophosphate, tetrasodium pyrophosphate, tetrapotassium pyrophosphate, sodium tripolyphosphate, potassium tripolyphosphate, trisodium orthophosphate, tripotassium orthophosphate, and sodium and/or potassium formates, citrates, acetates, and tartrates, and mixtures of the above. In some embodiments, sodium chloride is especially preferred. The electrolyte level, when present, is generally about 0.2% to about 4.5% by weight of the composition.

Acidic materials can be added to the bar to control free alkalinity. A suitable example is citric acid added at a level of about 0.1% to about 3%.

Another desirable ingredient of the composition, for aesthetic purposes, is a pearlescent material, such as mica, titanium-dioxide coated mica, natural fish silver or heavy metal salts, such as bismuth oxychloride.

The bar soap compositions may also contain any of the conventional perfumes, dyes, and coloring agents generally used in commercially-marketed bars to improve the characteristics of such products. When present, such perfumes, dyes, and coloring agents comprise about 0.2% to about 5% by weight of the bar.

### Antiperspirant/Deodorant

In some embodiments, an oil adduct of the present invention may be included in an antiperspirant and/or deodorant composition. In some embodiments, an antiperspirant and/or deoderant composition may include an oil adduct in an amount of about 0.01 to about 2 weight %, or about 0.01 to about 0.5 weight %.

Antiperspirants and deodorants containing an oil adduct of the present invention may include the conventional antiperspirant and deodorant materials. Examples of suitable compositions methods of making are disclosed in EP 1339122B1, WO 2005/072694A1, U.S. Pat. No. 7,011,822, and WO 2004/000254.

An antiperspirant and/or deodorant may include any conventional form. Various product forms include sticks (especially gel/sticks), gels, soft solids, roll-ons, aerosols and creams. Of these various forms the sticks, gels, soft solids creams and roll-ons are made with a liquid base material incorporating a solidifying agent and/or gelling agent and/or thickening agent.

### Antiperspirant Actives

An antiperspirant active can be selected from any of the known antiperspirant active materials. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex PG, aluminum chlorohydrex PEG, aluminum dichlorohydrex PG, and aluminum dichlorohydrex PEG. The aluminum-containing materials can be commonly referred to as antiperspirant active aluminum salts. Generally, the foregoing metal antiperspirant active materials are antiperspirant active metal salts. In some embodiments, antiperspirant compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as aluminum nitratohydrate and its combination with zirconyl hydroxychlorides and nitrides, or aluminum-stannous chlorohydrates, can be incorporated as an antiperspirant active ingredient in antiperspirant compositions according to the present invention.

Types of antiperspirant actives include aluminum zirconium trichlorohydrex and aluminum zirconium tetrachlorohydrex either with or without glycine. One antiperspirant active is aluminum trichlorohydrex gly such as AZZ-902 SUF (from Reheis Inc. Berkley Heights, N.J.); Westchlor 30BDM XF (from Westwood Chemical Co. Middletown, N.Y.). Tetrachlorohydrex salts include AZP 902 SUF from Reheis and Westchlor 35BDM XF from Westwood. Any of these salts can be processed to obtain 98% of the particles less than 10 microns in size; 95% of the particles less than 10 microns in size; 90% of the particles less than 10 microns in size; or 85% of the particles less than 10 microns in size.

In some embodiments, antiperspirant actives can be incorporated in the compositions of the present invention which include the enhanced efficacy aluminum salts and the enhanced efficacy aluminum/zirconium salt-glycine materials, having enhanced efficacy due to improved molecular distribution, known in the art and discussed, for example, in PCT No. WO92/19221. Actives include Westchlor A2Z 4105 aluminum zirconium tetrachlorohydrex gly propylene glycol complex, (from Westwood Chemical Corporation, Middletown, N.Y.); Westchlor ZR 35B aluminum zirconium tetrachlorhydrex gly, and Rezal 36 GP and AZP 902 aluminum zirconium tetrachlorhydrex gly both from Reheis, Berkeley Heights, N.J. as well as Rezal AZZ 908 from Reheis. In general, the metal:chloride mole ratio is in the range of 2.1-0.9:1 for such salts.

Actives of special interest because they form low RI solutions include: Westchlor Zr 35BX3 (30-35% actives in water) from Westwood Chemical Company, Middletown, N.Y.; Rezal 36G (46% in water) from Reheis Inc., Berkeley Heights, N.J.; Summit AZG-368 (28-32% in water) from Summit Research Labs, Huguenot, N.Y.; Reach 301 (39% in water) from Reheis Inc.; and aluminum chloride (28% in water) which may be obtained from several sources. In general, the metal:chloride mole ratio is approximately 1.4:1 for such salts.

In one type of salt, an aluminum zirconium tetra salt with glycine is used wherein aluminum zirconium tetrachlorohydrex glycine salt having a metal to chloride ratio in the range of 0.9-1.2:1 (especially in the range of 0.9-1.1:1 or in the range of 0.9-1.0:1); and a glycine:zirconium mole ratio greater than 1.3:1, or greater than 1.4:1.

According to some embodiments, antiperspirant actives may be incorporated into compositions in amounts in the range of 0.1-25% of the final composition, but the amount used will depend on the formulation of the composition. For example, at amounts in the lower end of the broader range (for example, 0.1-10% on an actives basis), a deodorant effect may be observed. At lower levels the antiperspirant active material will not substantially reduce the flow of perspiration, but will reduce malodor, for example, by acting as an antimicrobial material. At amounts of 10-25% (on an actives basis) such as 15-25%, by weight, of the total weight of the composition, an antiperspirant effect may be observed.

### Emollient

In some embodiments, an antiperspirant and/or deoderant may include an emollient. Emollients are a known class of materials in this art, imparting a soothing effect to the skin. These are ingredients which help to maintain the soft, smooth, and pliable appearance of the skin. Emollients are also known to reduce whitening on the skin and/or improve aesthetics. Examples of chemical classes from which suitable emollients can be found include:
(a) fats and oils which are the glyceryl esters of fatty acids, or triglycerides, normally found in animal and plant tissues, including those which have been hydrogenated to reduce or eliminate unsaturation. Also included are synthetically prepared esters of glycerin and fatty acids. Isolated and purified fatty acids can be esterified with glycerin to yield mono-, di-, and triglycerides. These are relatively pure fats which differ only slightly from the fats and oils found in nature. The general structure may be represented by the following formula:

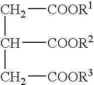

wherein each of R¹, R², and R³ may be the same or different and have a carbon chain length (saturated or unsaturated) of 7 to 30. Specific examples include peanut oil, sesame oil, avocado oil, coconut, cocoa butter, almond oil, safflower oil, corn oil, cotton seed oil, castor oil, hydrogenated castor oil, olive oil, jojoba oil, cod liver oil, palm oil, soybean oil, wheat germ oil, linseed oil, and sunflower seed oil;
(b) hydrocarbons which are a group of compounds containing only carbon and hydrogen. These are derived from petrochemicals. Their structures can vary widely and include aliphatic, alicyclic and aromatic compounds. Specific examples include paraffin, petrolatum, hydrogenated polyisobutene, and mineral oil.
(c) esters which chemically, are the covalent compounds formed between acids and alcohols. Esters can be formed from almost all acids (carboxylic and inorganic) and any alcohol. Esters here are derived from carboxylic acids and an alcohol. The general structure would be R⁴CO—OR⁵. The chain length for R⁴ and R⁵ can be 7 to 30 and can be saturated or unsaturated, straight chained or branched. Specific examples include isopropyl myristate, isopropyl palmitate, isopropyl stearate, isopropyl isostearate, butyl stearate, octyl stearate, hexyl laurate, cetyl stearate, diisopropyl adipate, isodecyl oleate, diisopropyl sebacate, isostearyl lactate. C₁₂₋₁₅ alkyl benzoates, myreth-3 myristate, dioctyl malate, neopentyl glycol diheptanoate, neopentyl glycol dioctanoate, dipropylene glycol dibenzoate, C₁₂₋₁₅ alcohols lactate, isohexyl decanoate, isohexyl caprate, diethylene glycol dioctanoate, octyl isononanoate, isodecyl octanoate, diethylene glycol diisononanoate, isononyl isononanoate, isostearyl isostearate, behenyl behenate. C₁₂₋₁₅ alkyl fumarate, laureth-2 benzoate, propylene glycol isoceteth-3 acetate, propylene glycol ceteth-3 acetate, octyldodecyl myristate, cetyl ricinoleate, myristyl myristate.
(d) saturated and unsaturated fatty acids which are the carboxylic acids obtained by hydrolysis of animal or vegetable fats and oils. These have general structure R⁶COOH with the R⁶ group having a carbon chain length between 7 and 30, straight chain or branched. Specific examples include lauric, myristic, palmitic, stearic, oleic, linoleic and behenic acid.
(e) saturated and unsaturated fatty alcohols (including guerbet alcohols) with general structure R⁷CH₂OH where R⁷ can be straight or branched and have carbon length of 7 to 30. Specific examples include lauryl, myristyl, cetyl, isocetyl, stearyl, isostearyl, oleyl, ricinoleyl and erucyl alcohol;
(f) lanolin and its derivatives which are a complex esterified mixture of high molecular weight esters of (hydroxylated) fatty acids with aliphatic and alicyclic alcohols and sterols. General structures would include R⁸CH₂—(OCH₂CH₂)ₙOH where R⁸ represents the fatty groups derived from lanolin and n=5 to 75 or R⁹CO—(OCH₂CH₂)ₙOH where R⁹CO—represents the fatty acids derived from lanolin and n=5 to 100. Specific examples include lanolin, lanolin oil, lanolin wax, lanolin alcohols, lanolin fatty acids, isopropyl lanolate, ethoxylated lanolin and acetylated lanolin alcohols.
(g) alkoxylated alcohols wherein the alcohol portion is selected from aliphatic alcohols having 2-18 or 4-18 carbons, and the alkylene portion is selected from the group consisting of ethylene oxide, and propylene oxide having a number of alkylene oxide units of 2-53 or 2-15. Specific examples include PPG-14 butyl ether, PPG-53 butyl ether, and PPG-3 myristyl ether.
(h) silicones and silanes which are organo-substituted polysiloxanes which are selected from polymers of silicon/oxygen having general structures: (1) (R¹⁰)₃SiO(Si(R¹¹)₂O)ₓSi(R¹²)₃ where R¹⁰, R¹¹ and R¹² can be the same or different and are each independently selected from the group consisting of phenyl and C₁-C₆₀ alkyl; (2) HO(R¹⁴)₂SiO(Si(R¹⁵)₂O)ₓSi(R¹⁶)₂OH, where R¹⁴, R¹⁵ and R¹⁶ can be the same or different and are each independently selected from the group consisting of phenyl and C₁-C₆₀ alkyl; or (3) organo substituted silicon compounds of formula R¹⁷Si(R¹⁸)OSiR¹⁹ which are not polymeric where R¹⁷, R¹⁸ and R¹⁹ can be the same or different and are each independently selected from the group consisting of phenyl and C₁-C₆₀ alkyl optionally with one or both of the terminal R groups also containing an hydroxyl group. Specific examples include dimethicone (for example, dimethicone having a viscosity of 0.5-1.5 centistokes), dimethiconol behenate, C₃₀₋₄₅ alkyl methicone, stearoxytrimethylsilane, phenyl trimethicone and stearyl dimethicone.
(i) mixtures and blends of two or more of the foregoing.

Emollients of special interest include C₁₂₋₁₅ alkyl benzoate (FINSOLV TN from Finetex Inc. Elmwood Park, N.J.); phenyltrimethicone, isopropyl myristate; and neopentyl glycol diheptanoate.

The emollient or emollient mixture or blend thereof incorporated in compositions according to the present invention can, illustratively, be included in amounts of 0.5-50%, preferably 1-25%, more preferably 3-12%, by weight, of the total weight of the composition. One elastomer of interest is DC-9040 from Dow Corning Corporation.

### Antimicrobial

In some embodiments, an antiperspirant and/or deodorant composition may include antimicrobial agents. Suitable antimicrobial agents include, for example, bacteriostatic quaternary ammonium compounds such as 2-amino-2-methyl-1-propanol (AMP), cetyl-trimethylammonium bromide, cetyl pyridinium chloride. 2,4,4′-trichloro-2′-hydroxydiphenylether (Triclosan), N-(4-chlorophenyl)-N′-(3,4-dichlorophenyl)urea (Triclocarban), silver halides, octoxyglycerin (Sensiva™ SC 50) and various zinc salts (for example, zinc ricinoleate). The bacteriostat can, illustratively, be included in the composition in an amount of 0-5%, or 0.01-1.0% by weight, of the total weight of the composition. Triclosan, can illustratively be included in an amount of 0.05% to about 0.5% by weight, of the total weight of the composition.

### Additional Materials

A variety of fragrances can be used in these compositions if a scented product is desired. Fragrances can be used in an amount in the range of 0-5%, 0.01-2.0%, and, for example, at a level of 1%.

Masking agents can be used in an amount of 0.05-5.0% (or 0.05-2%) by weight based on the total weight of the composition if an unscented product is desired.

Other various optional components include coloring agents, soothing agents (such as aloe and its derivatives), opacificers, etc. in types and amounts conventionally used for such products, some of which have already been described above.

### Additional Compositions

Oil adducts of the present invention may be included in any suitable personal care product such as cosmetics, lotions, shampoos, and liquid hand soaps. Examples of suitable cosmetic compositions and methods of making may be found in EP 1224928B1, EP 1212035B1. WO 01/47479A2, and U.S. Pat. No. 4,009,254. Examples of suitable lotion compositions and methods of making may be found in U.S. Pat. No. 5,385,729 and U.S. Pat. No. 6,730,310. Examples of suitable shampoo compositions and methods of making may be found in EP 0413417B1, EP 1119339B, EP 0417042B1, U.S. Pat. No. 5,346,642, and U.S. Pat. No. 5,213,716. Examples of suitable liquid hand soap compositions and methods of making may be found in EP 1175201B1, WO 00/66079 and EP 0584877A2.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights are averages.

### Examples 1, 2, and 3

A shower gel was prepared with castor oil lactate, according to the following formulas: The compositions were prepared by mixing of the ingredients for a standard shower gel.

| | Example 1 | Example 2 | Example 3 |
|---|---|---|---|
| Ingredient | Weight % | Weight % | Weight % |
| C₁₀-C₁₆ alcohol ethoxylate, sulfated, | 6.89 | 6.89 | 6.89 |
| sodium salt | | | |
| Glycerin | 5.96 | 5.96 | 5.96 |
| Cocamidopropyl betaine | 5.18 | 5.18 | 5.18 |
| Glycol distearate | 0.88 | 0.88 | 0.88 |
| Sodium Chloride | 0.85 | 0.85 | 0.85 |
| Alkyl polyglucoside | 0.66 | 0.66 | 0.66 |
| Castor oil lactate | 0.5-0.75 | 0 | 0 |
| Castor oil glycolate | 0 | 0.5-0.75 | 0 |
| Castor oil salicylate | 0 | 0 | 0.5-0.75 |
| Laureth-4 | 0.53 | 0.53 | 0.53 |
| Lauryl polyglucoside | 0.28 | 0.28 | 0.28 |
| Polyquaternium-7 | 0.17 | 0.17 | 0.17 |
| Tetrasodium EDTA | 0.07 | 0.07 | 0.07 |
| Fatty alcohol, C₁₀₋₁₆. ethoxylated | 0.03 | 0.03 | 0.03 |
| Formaldehyde | 0.02 | 0.02 | 0.02 |
| Citric acid | 0.02 | 0.02 | 0.02 |
| Formic acid | 0.01 | 0.01 | 0.01 |
| Sodium sulfate | 0.01 | 0.01 | 0.01 |
| Disodium hydrogenorthophosphate | 0.01 | 0.01 | 0.01 |
| Water and Minors | Q.S. | Q.S. | Q.S. |
| Total | 100.00 | 100.00 | 100.00 |

What is claimed:

1. A composition of matter comprising an oil adduct of an oil capable of undergoing an adduct reaction and at least one addition material chosen from an alpha hydroxy acid and a beta hydroxy acid.

2. The composition of claim 1, wherein the oil is castor oil.

3. The composition of claim 1, wherein the addition material is at least one material chosen from lactic acid, glycolic acid, lactobionic acid, carnitine, and salicylic acid.

4. The composition of claim 1, wherein the addition material is lactic acid.

5. The composition of claim 1, wherein the addition material is salicylic acid.

6. The composition of claim 1, wherein the addition material is glycolic acid.

7. The composition of claim 1, wherein the oil is castor oil and the addition material is lactic acid.

8. The composition of claim 1 further comprising a surfactant to form a cleansing composition.

9. The composition of claim 4, wherein the oil adduct is present in an amount of about 0.1 wt % to about 5 wt %.

10. The composition of claim 1 further comprising a soap to form a bar soap.

11. The composition of claim 1 further comprising an antiperspirant salt and/or deodorant.

12. A method of forming the oil adduct of claim 1, comprising adducting the oil with the addition material.
